(19) United States
(12) Patent Application Publication
(10) Pub. No.: US 2023/0136813 A1
(43) Pub. Date: May 4, 2023

(54) PATIENT-SPECIFIC ARTHROPLASTY DEVICES AND ASSOCIATED SYSTEMS AND METHODS

(71) Applicant: Carlsmed, Inc., Carlsbad, CA (US)

(72) Inventors: Michael J. Cordonnier, Carlsbad, CA (US); Niall Patrick Casey, Carlsbad, CA (US); Christopher K. Ames, Carlsbad, CA (US)

(21) Appl. No.: 17/518,524

(22) Filed: Nov. 3, 2021

(51) Int. Cl. A61F 2/46; A61F 2/44; G16H 10/60; G16H 20/40

(52) U.S. Cl. CPC A61F 2/46; A61F 2/4425; G16H 10/60; G16H 20/40; A61F 2002/4633; A61F 2002/30649

(57) ABSTRACT

The present technology is directed to patient-specific medical devices, such as patient-specific implants, and systems and methods for designing the same. For example, the present technology includes patient-specific arthroplasty devices for use in restoring and/or improving joint function in general, and, in particular, for restoring and/or improving function of intervertebral joints. The present technology also provides methods for designing, manufacturing, and/or providing patient-specific arthroplasty devices and systems.

### TECHNICAL FIELD

The present disclosure is generally related to orthopedic implants, and more particularly to patient-specific arthroplasty devices.

### BACKGROUND

Orthopedic implants are used to correct numerous different maladies in a variety of contexts, including total joint reconstruction (arthroplasty), spine surgery, hand surgery, shoulder and elbow surgery, skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include arthritis, osteoarthritis, lumbar degenerative disc disease or cervical degenerative disc disease, lumbar spinal stenosis, and cervical spinal stenosis.

In some instances, arthroplasty implants (e.g., arthroplasty devices) are implanted into a patient’s spine to decompress, stabilize, and/or improve motion of the spine. Arthroplasty procedures may be performed on cervical, lumbar, or thoracic regions of the spine. For example, arthroplasty devices can be used to improve or restore the relative position of vertebrae and provide relative motion between vertebral bodies of the spine.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Non-limiting and non-exhaustive descriptions are described with reference to the following drawings. The components in the figures are not necessarily to scale, emphasis instead being placed upon illustrating principles.

FIG. 1A is a side view of a portion of a human skeleton illustrating a plurality of patient-specific arthroplasty devices positioned between vertebral bodies and configured in accordance with select embodiments of the present technology.

FIG. 1B is a side view of a segment of a cervical vertebral column of a human patient and illustrates the center of rotation (COR) locations of the vertebral bodies in accordance with select embodiments of the present technology.

FIGS. 2A and 2B are lateral and anterior views, respectively, of a patient-specific arthroplasty device positioned between vertebral bodies in a first configuration and configured in accordance with select embodiments of the present technology.

FIG. 2C is a lateral view of the patient-specific arthroplasty device of FIG. 2A in a second configuration.

FIG. 2D is a lateral view of the patient-specific arthroplasty device of FIG. 2A in a third configuration.

FIG. 3 is an anterior view of a patient-specific arthroplasty system positioned between vertebral bodies and configured in accordance with select embodiments of the present technology.

FIG. 4A is a schematic illustration of a patient-specific arthroplasty device in a first configuration and configured in accordance with select embodiments of the present technology.

FIG. 4B is a schematic illustration of the patient-specific arthroplasty device of FIG. 4A in a second configuration.

FIG. 5 is a schematic illustration of a patient-specific arthroplasty device configured in accordance with select embodiments of the present technology.

FIG. 6 is a network connection diagram illustrating a computing system for providing patient-specific devices in accordance with embodiments of the present technology.

FIG. 7 illustrates a computing device suitable for use in connection with the computing system of FIG. 6 in accordance with select embodiments of the present technology.

FIG. 8 is a flow diagram illustrating a method for designing a patient-specific arthroplasty device or a system including two or more patient-specific arthroplasty devices in accordance with select embodiments of the present technology.

### DETAILED DESCRIPTION

### Overview of Technology

The present technology is directed to patient-specific medical devices, such as patient-specific implants, and systems and methods for designing the same. For example, the present technology includes patient-specific arthroplasty devices for use in restoring and/or improving joint function in general, and, in particular, for restoring and/or improving function of intervertebral joints. The present technology also provides methods for designing, manufacturing, and/or providing patient-specific arthroplasty devices and systems.

The patient-specific arthroplasty devices described herein can be specifically tailored to achieve one or more desired patient outcomes following implantation of the patient-specific arthroplasty devices into the patient. For example, the patient-specific arthroplasty devices may provide correction to the patient’s anatomy while also maintaining or improving movement of the patient’s spine. For example, the patient-specific arthroplasty devices can be configured to restore and/or improve rotational and/or translational motion of the patient’s spine. As another example, the patient-specific arthroplasty devices can be configured to provide compression between vertebrae of the patient’s spine. In some embodiments, the patient-specific arthroplasty devices are designed to maintain and/or achieve a pre-determined patient-specific intervertebral center of rotation (COR) when the devices are implanted. As used herein, intervertebral COR of a specific region of the spine corresponds to a point around which the spinal region rotates. The arthroplasty devices of the present technology may further be configured to provide for improved and/or optimal sagittal and coronal balance in the patient’s spine.

Accordingly, in some embodiments, the patient-specific arthroplasty devices can improve or restore a relative position of adjacent vertebrae while also permitting a desired range of motion between the adjacent vertebrae. For example, patient-specific arthroplasty devices and systems are configured to include mobility elements that, when implanted between two or more vertebrae of a vertebral segment of the patient’s spine, allow translational and/or rotational movement of the vertebral segment as well as compression of the vertebral segment. The mobility elements and features associated with the mobility elements can be designed based on pre-determined optimal CORs for the vertebral bodies associated with the arthroplasty device. Furthermore, the patient-specific arthroplasty devices can have design characteristics (e.g., shape, topography, etc.) configured to mate with the particular patient’s anatomy to reduce the risk of migration and further improve patient outcomes. In some embodiments, for example, endplates of the arthroplasty devices are designed to match the vertebral body end-plates of the patient to form a substantially gapless interface therebetween.

In some embodiments, the patient-specific arthroplasty devices described herein are designed using patient data to enhance the performance of the device. The patient data can include image data (e.g., anatomy data), kinematic data (e.g., motion data), medical history, patient information, and the like. The anatomy data can include the geometry and/or topography of anatomical features, spacing between adjacent anatomical features, characteristics (e.g., tissue characteristics), and the like. The kinematic data can include a range of motion data (e.g., target operational range of motion data, pre-surgery operational range of motion data, etc.), target COR locations at specific vertebral bodies, and other kinematic characteristics. The kinematic data can be collected by performing motion studies, modeling the motion of joints using a software module, or other techniques. The kinematic data can be associated with a subject joint or motion segment.

In some embodiments, the patient-specific arthroplasty devices described herein are designed using one or more design criteria, in addition to or in lieu of the patient data. The design criteria can include, but is not limited to, a target range of motion, a target COR, a target vertebral spacing (e.g., minimum vertebral body spacing), vertebral end-plate topography, implantation procedures (e.g., access path or procedure), expected service life, patient-specific needs, regulatory requirements, etc. For example, the patient-specific arthroplasty devices can be configured to match the intervertebral space, topography of vertebral end-plates, kinematics of subject joints, or combinations thereof. In some procedures, the patient-specific arthroplasty devices can be configured to maintain rotational and/or translational motion of the spine to reduce the risk of complications. In other procedures, the patient-specific arthroplasty devices can be configured to increase the motion of the spine. In some embodiments, the present technology incorporates predictive analytics, machine learning, neural networks, and/or artificial intelligence (Al) to define improved or optimal surgical interventions and/or implant designs in order to achieve the desired efficacy. For example, the patient data can be used to generate a patient-specific arthroplasty devices design for providing one or more joint characteristics (e.g., range of motion, disc height, etc.).

In accordance with some embodiments, an arthroplasty system includes a patient-specific arthroplasty device for insertion in a patient’s spine. The patient-specific arthroplasty device includes a first end-plate having a first patient-specific topography and a second end-plate having a second patient-specific topography. The patient-specific arthroplasty device also includes a mobility element disposed between the first end-plate and the second end-plate. The mobility element is configured for allowing movement of the first end-plate and the second end-plate relative to each other. A position of the mobility element relative to the first end-plate and the second end-plate is designed to maintain and/or achieve a pre-determined patient-specific center of rotation when the patient-specific arthroplasty device is implanted in the patient’s spine. The pre-determined patient-specific center of rotation is based on a designed target configuration and/or desired target kinematic parameters for a region of the patient’s spine at which the patient-specific arthroplasty device is to be inserted.

In some embodiments, the present technology provides methods for providing patient-specific arthroplasty devices. In a particular embodiment, the method includes obtaining image data of one or more regions of a patient’s spine that depicts a native anatomical configuration of the one or more regions. The method further includes obtaining kinematic data associated with the one or more regions of the patient’s spine. The kinematic data can include values for one or more kinematic parameters, such as center of rotation locations, range of motion, angle of bend, angle of rotation, displacement, flexion, extension, flexion/extension arc, lateral bending, left/right bending arc, and/or axial rotation. The method further includes determining a target operational configuration different than the native anatomical configuration. A patient-specific arthroplasty device is then designed based on the target operational configuration and the kinematic parameter values. In particular, the patient-specific arthroplasty device is designed such that, when it is implanted in the patient, the patient-specific arthroplasty device provides the target operational configuration while maintaining or improving the kinematic parameters. For example, the designed patient-specific arthroplasty device provides restored or improved rotational movement of a segment of the spine with respect to pre-determined optimal center of rotation locations.

In another particular embodiment, a computer-implemented method in accordance with the present technology includes receiving image data of one or more regions of a patient’s spine that depicts a native anatomical configuration of the one or more regions. The method further includes measuring one or more kinematic parameters associated with the one or more regions and determining a target operational configuration different than the native anatomical configuration. A patient-specific implant is then designed based on the target operational configuration and the measured kinematic parameters. In particular, the patient-specific implant is designed such that, when it is implanted in the patient, the patient-specific implant provides target rotational and/or translational movement of the patient’s spine for the region where the patient-specific arthroplasty device is implanted.

In some embodiments, the computer-implemented method for designing a patient-specific implant uses acquired patient data. The patient data can include one or more images, kinematic data, physician inputted data, or the like. The images can show native anatomical features of the patient. The kinematic data can be associated with the one or more regions and can include one or more specific values for various kinematic parameters. The kinematic parameters can include a range of motion, angle of bend, angle of rotation, flexion/extension arcs, left/right bending arcs, lateral bending, displacement, and other parameters related to flexion, extension, bending, axial rotation, etc., and under a variety of conditions (e.g., load-bearing, non-load bearing, etc.). The values for kinematic parameters can be determined based on images of the patient in different positions, measuring body position/motion, or the like. In some embodiments, the values for the kinematic parameters can be compared to target values for the kinematic parameters. The target values can be a target range of motion, angle of bend, angle of rotation, center of rotation, displacement, and/or other parameters related to flexion, extension, bending, axial rotation, or the like. For example, the target values can include a target rotational movement, a target translational movement, and/or target compression. A target operational configuration for one or more regions of the patient can also be determined. The target operational configuration can include an adjustment to one or more kinematic parameters relative to the native parameters, including, but not limited to, and adjustment to the spacing between vertebral bodies, relative movement of the vertebral bodies, orientation of vertebral bodies, alignment of two or more vertebral bodies, lumbar lordosis, Cobb angle(s), pelvic incidence, disc height, segment flexibility, rotational displacement, and the like. At least a portion of the patient-specific arthroplasty devices can be designed based at least in part on the target operational configuration and the kinematic parameter values.

The computer-implemented method can include the identification of anatomical features that impair body motion. The computer-implemented method can generate kinematic algorithms based on the identified features and can design the patient-specific arthroplasty device based on the kinematic algorithms to maintain a threshold amount of spine movement (e.g., movement of the cervical spine), maintain pre-treatment spine movement, and/or improve spine movement. In some embodiments, a predicted amount of movement can be determined using one or more predictive models. A designer can update the predictive models. Secondary procedures can be performed on the identified anatomical features (e.g., stenosis, enlarged facet joints, bony overgrowths, loss of cartilage, etc.) to further enhance or affect spine movement. The kinematic algorithms can model one or more segments of the spine as a kinematic chain of links using constraints and boundary conditions to model segment configuration, movements, range of motion, degrees of freedom, etc. For example, a fixed link can represent fused vertebrae along the segment. Images of the patient’s body in different positions and other patient data (including the present patient and/or prior patients) can be used to automatically generate a virtual model for two- or three-dimensional analysis.

In accordance with some embodiments, a computer-implemented method for designing a patient-specific arthroplasty device includes obtaining patient data. The patient data includes image data of a region of a patient’s spine. The image data depicts a native anatomical configuration of the one or more regions. The patient data also includes kinematic data associated with the one or more regions of a patient’s spine. The kinematic data includes values for one or more kinematic parameters. The method includes determining, based on the obtained image data and the kinematic data, a target configuration for a region of the patient’s spine where the patient-specific arthroplasty device is to be inserted. The target configuration includes a target movement of the patient’s spine and a pre-determined patient-specific center of rotation. The method further includes designing a patient-specific arthroplasty device based on the target configuration. The patient-specific arthroplasty device includes a first end-plate having a first patient-specific topography, a second end-plate having a second patient-specific topography, and a mobility element disposed between the first end-plate and the second end-plate. A position of the mobility element relative to the first end-plate and the second end-plate is designed to maintain and/or achieve the pre-determined patient-specific center of rotation when the device is implanted in the patient’s spine.

The patient-specific arthroplasty devices described herein are expected to provide a number of advantages over conventional artificial arthroplasty devices. For example, the patient-specific arthroplasty devices described herein can provide for implants that are personalized to achieve ideal segmental lordosis, decompression, motion, and center of rotation needs for each individual patient. The implants are designed for each individual prior to surgery based on the individual patient’s anatomy, medical conditions, age, gender, activity level, etc., to ensure optimal, individualized movement of the spine.

Additionally, the patient-specific arthroplasty devices described herein can reduce the number of surgical steps required during an implant procedure. Conventional spinal implants, including arthroplasty devices, are manufactured in standard shapes and sizes and with standard flexibilities. Minimal consideration is paid to implant size and other characteristics before an implant procedure. Instead, during an implant procedure and with a patient’s spine exposed, a surgeon will select a specific implant from a surgical kit containing a variety of sizes and shapes. Typically, the surgeon selects the implant size through a technique known as “trialing,” during which the surgeon uses a series of incrementally sized implant proxies or subcomponents to determine the appropriate implant size and shape. Trialing can be a timely process, and the surgeon typically only focuses on the posterior height and sagittal angle of the implants, while largely ignoring the lateral heights and coronal angle of the implants. Using the present technology, the trailing process can be eliminated because the patient-specific arthroplasty devices described herein have already been properly sized for the patient.

The patient-specific arthroplasty devices can further facilitate proper placement and be designed to reduce the number of implant failures by optimizing fit, mobility, flexibility, and/or other characteristics of the implant. Improper placement or sizing of spinal implants can result in implant failures. For example, if an arthroplasty device is improperly placed, it can lead to issues with other joints of the motion segment. In one instance, if an arthroplasty device is not placed in the appropriate location or sized correctly, the associated facet joints can become over-stressed and suffer degeneration. Moreover, insufficient contact and load transfer between the vertebrae and the implant can produce inadequate fixation between the implant and anatomy. Inadequate fixation can allow the implant to move relative to the vertebrae, which can lead to improper placement of the implant. Furthermore, insufficient contact area or fixation between the interbody implant and the vertebrae can result in micro- and/or macro-motions that can reduce the opportunity for bone growth and fusion to the implant to occur. The patient-specific arthroplasty devices described herein can therefore be configured to facilitate placement to limit stresses (e.g., limit stresses in the vertebral body, facet joints, etc.), enhance fixation, provide a relatively large contact area, or other design criteria. As one skilled in the art will appreciate from the disclosure herein, the arthroplasty device may provide additional advantages over conventional implants and implant procedures, regardless of whether such problems are described herein.

The present technology thus provides systems and methods for designing “patient-specific” or “personalized” medical devices, such as patient-specific arthroplasty devices, that are expected to mitigate at least some of the foregoing disadvantages of conventional stock devices. In particular, the present technology provides systems and methods for designing patient-specific arthroplasty devices that are optimized for the patient’s particular characteristics (e.g., condition, anatomy, pathology, medical history, activity level, age, gender, etc.). For example, the patient-specific arthroplasty devices can be designed and manufactured specifically for the particular patient, rather than being an off-the-shelf implant. However, it shall be appreciated that a patient-specific or personalized medical implant can include one or more components that are non-patient-specific, and/or can be used with an instrument or tool that is non-patient-specific. Personalized implant designs can be used to manufacture or select patient-specific technologies, including medical implants, instruments, and/or surgical kits. For example, a personalized surgical kit can include one or more patient-specific implants, patient-specific instruments, non-patient-specific technology (e.g., standard instruments, devices, etc.), instructions for use, patient-specific treatment plan information, or a combination thereof.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

### Patient-Specific Implants

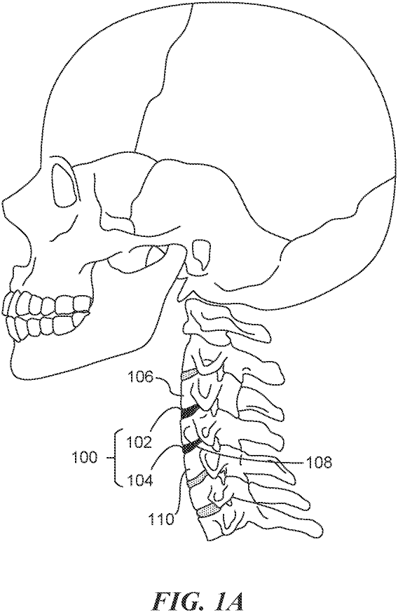

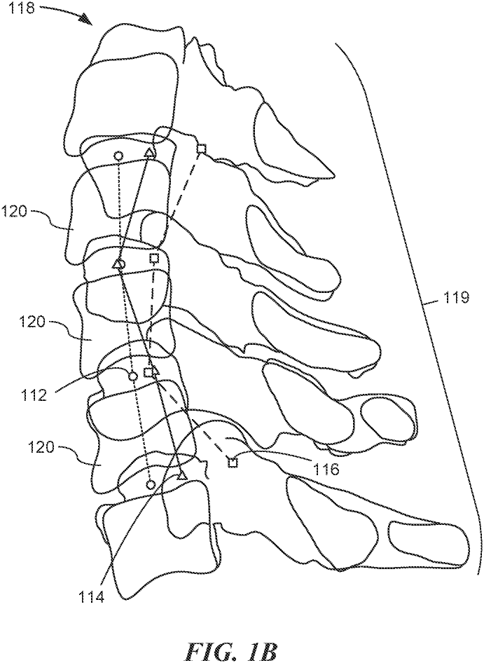

FIG. 1A is a schematic illustration of patient-specific arthroplasty devices 102 and 104 (referred to as “devices”) positioned between vertebral bodies of a patient’s spine in accordance with select embodiments of the present technology. In FIG. 1A, the device 102 is implanted between vertebral bodies 106 and 108 and the device 104 is implanted between vertebral bodies 108 and 110. Jointly, the devices 102 and 104 form an arthroplasty system 100. In FIG. 1A, the arthroplasty system 100 includes two arthroplasty devices (e.g., devices 102 and 104), but it is understood that the arthroplasty system 100 may also include three, four, five, or more devices based on the patient’s need. In some embodiments, only a single device (e.g., the device 102 or the device 104) is included. In some embodiments, the devices 102 and 104 are positioned to replace adjacent intervertebral discs, as shown in FIG. 1A. The devices 102 and 104 may also be positioned so that there is one or more intervertebral discs between them that are not replaced with implants (e.g., the device 102 is positioned between vertebral bodies C3 and C4, and the device 104 is positioned between vertebral bodies C5 and C6, such that the native disc between vertebral bodies C4 and C5 remains intact). Moreover, although FIG. 1A illustrates the devices 102, 104 positioned within a cervical region of the patient’s spine, in other embodiments some or all of the system 100 may be positioned in other spinal regions, including thoracic (e.g., between vertebral bodies T1-T12) and/or lumbar (e.g., between vertebral bodies L1-L5) regions. In some embodiments, the system 100 may include at least one device in a first spinal region (e.g., cervical) and a second device in a second spinal region (e.g., thoracic).

FIG. 1B is a side view of a segment 119 (e.g., a cervical segment) of a spine 118 of a human patient, and illustrates intervertebral center of rotation (COR) locations of vertebral bodies 120. As explained above, intervertebral COR corresponds to a point or points around which two adjacent vertebral bodies rotate and/or translate relative to one another. In some embodiments, the COR can be at the geometric center between adjacent vertebral bodies. In other embodiments, the COR can be offset from the geometric center between vertebral bodies. For example, in FIG. 1B, spheres 112 represent estimated geometric centers of each vertebral body, triangles 114 represent CORs of each vertebral body during flexion-extension motion (e.g., a patient bending her neck forward and backward), and squares 116 represent pre-determined CORs of each intervertebral body during a left-right rotational motion (e.g., the patient turning her head between left and right sides). As shown, the positions of the CORs relative to the geometric centers of the vertebrae body vary along the spine. Similarly, the CORs for the different types of movements of the spine, e.g., flexion-extension motion vs. left-right rotational motion, vary along the spine. The CORs for each vertebral bodies 120 are patient-specific and depend on, e.g., the patient’s anatomy, age, size, activity, the patient’s health conditions, and/or other parameters associated with the patient’s spine.

Of note, in some patients their actual COR(s) may be suboptimal (e.g., based on their anatomy, disc-health, bone-growth, inflammation, nerve compression, etc.), leading to pain, limited flexibility, or other symptoms. As set forth in detail below, the present technology therefore includes systems, device, and methods for designing patient-specific arthroplasty devices that can, among other things, adjust or correct the patient’s COR(s) such that, following surgical implantation of the devices, the patient’s COR(s) are improved and/or optimized relative to the patient’s native (e.g., pre-surgical) COR(s). As set forth in detail below, the present technology can determine native (e.g., pre-surgery) CORs for a specific patient based on imaging data of the patient’s spine, a three-dimensional model of the patient’s spine, kinematic tests, or the like, and can likewise determine a recommend adjustment to the patient’s COR(s) to provide post-surgical symptom improvement or relief. The systems described herein can then design patient-specific devices to achieve the corrected COR(s).

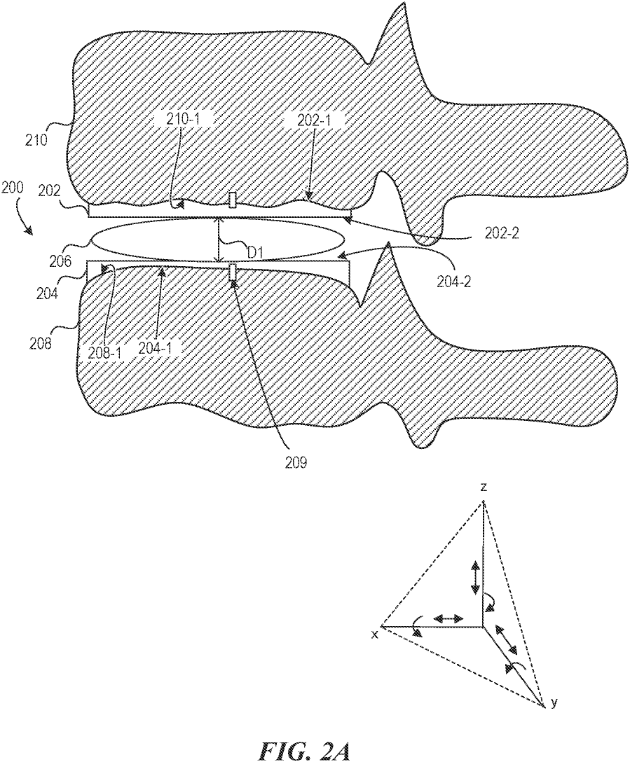

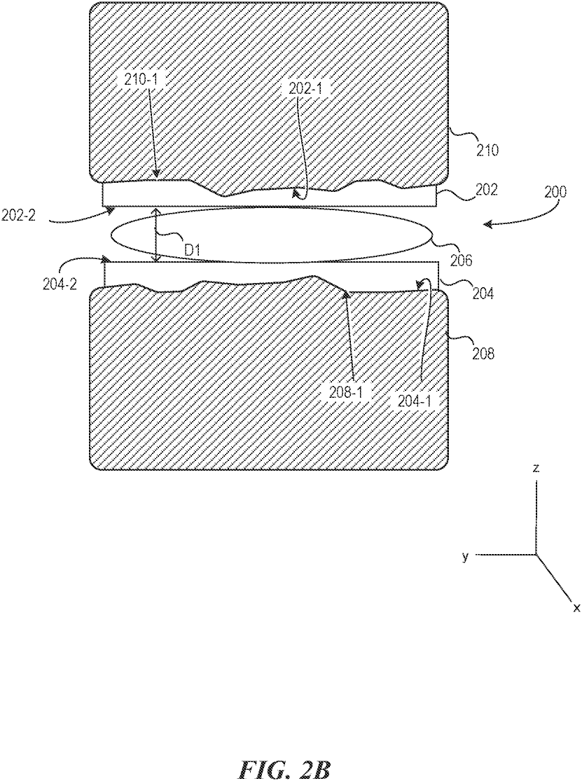

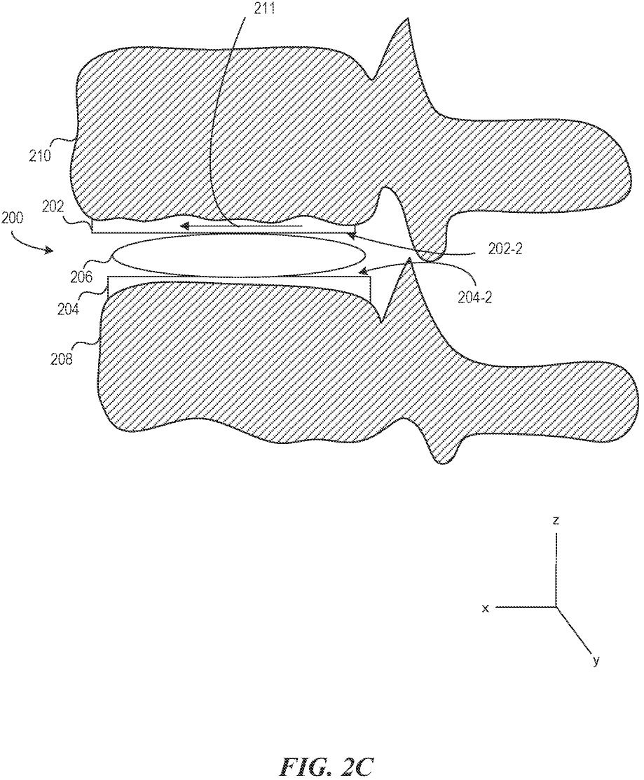

FIGS. 2A and 2B are schematic illustrations of a patient-specific arthroplasty device 200 (referred to as “device 200”) positioned between vertebral bodies 208 and 210 in a first configuration and configured in accordance with select embodiments of the present technology. FIG. 2A illustrates a cross-sectional view of the device 200 and vertebral bodies 208 and 210 from a side (e.g., lateral) view, and FIG. 2B illustrates a cross-sectional view of the device 200 and vertebral bodies 208 and 210 from a front (e.g., anterior) view. In some embodiments, the device 200 corresponds to the device 102 and/or the device 104 described with respect to FIG. 1A. The device 200 includes two end-plates: a first end-plate 202 (e.g., an upper end-plate) and a second end-plate 204 (e.g., a lower end-plate). The first end-plate 202 has a first (e.g. upper or outward facing) surface 202-1 configured to engage the vertebral body 210 and a second (e.g., lower or inward facing) surface 202-2. Likewise, the second end-plate 204 has a first (e.g., lower or outward facing) surface 204-1 configured to engage the vertebral body 208 and a second (e.g., upper or inward facing) surface 204-2. The device 200 also includes a mobility element 206 (e.g., a core) positioned between the second surface 202-2 of the first plate 202 and the second surface 204-2 of the second plate 204.

In some embodiments, the first and second end-plates 202 and 204 include one or more coupling elements 209 configured for coupling the first and second end-plates 202 and 204 to the vertebral bodies 210 and 208, respectively, and/or to the mobility element 206. The coupling elements 209 may include one or more types of coupling elements selected from a keel, a spike, a screw, or another type of coupling element known in the art. The coupling elements 209 are configured to provide stabilization of the arthroplasty device 200 when implanted between respective vertebral bodies of the spine. In some embodiments, the first and second end-plates 202 and 204 (and the coupling elements 209) are designed to allow direct engagement of the first and second end-plates 202 and 204 to the respective vertebral bodies 210 and 208. In some embodiments, the device 200 can provide for sagittal and/or coronal correction of the spine. For example, as described in detail below, the device 200 can be configured for correcting sagittal imbalance (i.e., a front-to-back imbalance in the spine), coronal deformity (i.e., a deviation from a midline in the coronal plane), and/or other deformities of the spine.

In some embodiments, the first surface 202-1 of the first end-plate 202 has a topography designed to mate with the topography of a corresponding first (e.g., lower) surface 210-1 of the vertebral body 210, and the first surface 204-1 of the second end-plate 204 has a topography designed to mate with the topography of a corresponding first (e.g., upper) surface 208-1 of the vertebral body 208. As used herein, the term “mate” can refer to the engagement of two surfaces with reduced and/or minimized space therebetween. For example, the first surface 202-1 of the first end-plate 202 can form a gapless or generally gapless interface with the lower surface 210-1 of the vertebral body 210. The first surface 204-1 of the second end-plate 204 can form a gapless or generally gapless interface with the upper surface 208-1 of the vertebral body 208. The surface profiles of the first end-plate 202 and the second end-plate 204 can therefore be designed based on the topography, shape, and features (e.g., ring apophysis, cortical rim, etc.) of the vertebral bodies with which they will interact once implanted. Without being bound by theory, this is expected to provide a relatively large contact area to limit stresses in the vertebral body 210 and the vertebral body 208, facilitate seating of the device 200, and/or limit or inhibit migration of the device 200. Accordingly, in some embodiments, the first end-plate 202 and the second end-plate 204 have different geometries and/or topographies to accommodate the different geometries and/or topographies of the first and second vertebral bodies 210, 208. Without being bound by theory, improving the fit between the end-plates and the vertebrae is expected to prevent and/or reduce instances of dynamic failure of the implanted devices (e.g., by reducing and/or preventing micro-motions of the device), and/or increase the efficacy of the devices.

The mobility element 206 positioned between the first and second end-plates 202 and 204 permits movement (e.g., rotational movement, translation movement, etc.) of the first and second end-plates 202 and 204 relative to each other. In FIGS. 2A and 2B, the mobility element 206 has a spherical shape defined by two curved surfaces. The mobility element 206 extends between and is at least partially in contact with the second surface 202-2 of first end-plate 202 and the second surface 204-2 of second end-plate 204. In some embodiments, the mobility element 206 includes a ceramic material, polymeric material, metallic material, or a combination thereof. In some embodiments, the mobility element 206 includes a viscoelastic material that allows for compression and decompression of the mobility element 206.

As set forth above, the mobility element 206 permits/enables movement of the first and second end-plates 202 and 204 (and thus movement of the vertebral bodies 210, 208) relative to each other. The first endplate 202 generally does not move relative to the vertebral body 210, and the second endplate 204 generally does not move relative to the vertebral body 208. Thus, movement of the first endplate 202 relative to the second endplate 204 generally includes a corresponding motion between the vertebral body 210 and the vertebral body 208.

In some embodiments, the mobility element 206 permits and/or enables translation movement, rotational movement, and/or both translation and rotational movement between the first and second endplates 202 and 204. Accordingly, the device 200 can permit and/or enable translational movement, rotational movement, and/or translational and rotational movement between the vertebral body 210 and the vertebral body 208. In some embodiments, the mobility element 206 enables the first and second end-plates 202 and 204 to rotate or pivot relative to each other along one or more of frontal, sagittal, and/or transverse planes, and/or translate relative to each other along one or more of the frontal, sagittal, and/or transverse planes. In some embodiments, the mobility element 206 can permit up to six degrees of freedom between the first and second endplates 202 and 204, thereby enabling up to six degrees of freedom between the vertebral bodies 208, 210, as illustrated by the xyz-coordinate in FIG. 2A. Accordingly, in some embodiments, the movement includes compression and decompression (e.g., translation in the z direction) so that a distance between the first and second end-plates 202 and 204 is adaptable. For example, the mobility element 206 can be compressed or decompressed such that a distance D1 between the vertebral bodies 208, 210 changes.

In some embodiments, the mobility element 206 is an unconstrained mobility element. An “unconstrained” mobility element allows rotational movement about the x, y, and z axis, and translational movement in the x, y, and z direction. For example, an unconstrained mobility element allows rotation as well as translation along all the planes illustrated in the xyz-coordinate of FIG. 2A (e.g., including rotational and translational movement along and about the x, y, and z directions). Accordingly, an unconstrained mobility element allows 6 degrees of freedom of movement (e.g., 3 rotational degrees of freedom and 3 translational degrees of freedom).

In some embodiments, the mobility element 206 is a semi-constrained mobility element. A “semi-constrained” mobility element allows some translational motion and/or rotational motion, but has fewer than six degrees of freedom. For example, a “semi-constrained” mobility element may allow rotational movement of the end-plates about the x, y, and z axis, as well as a translational movement in a defined direction, such as one or two of movement in the x, y, or z direction. As another example, a “semi-constrained” mobility element may allow rotational movement with three degrees of freedom, and prevent translation movement. In some embodiments, an arthroplasty system (e.g., the system 100 shown in FIG. 1A) can include a first device (e.g., the device 102) that has an unconstrained mobility element and thus permits 6 degrees of freedom of movement, and a second device (e.g., the device 104) that has an semi-constrained mobility element and thus permits fewer than 6 degrees of freedom of movement.

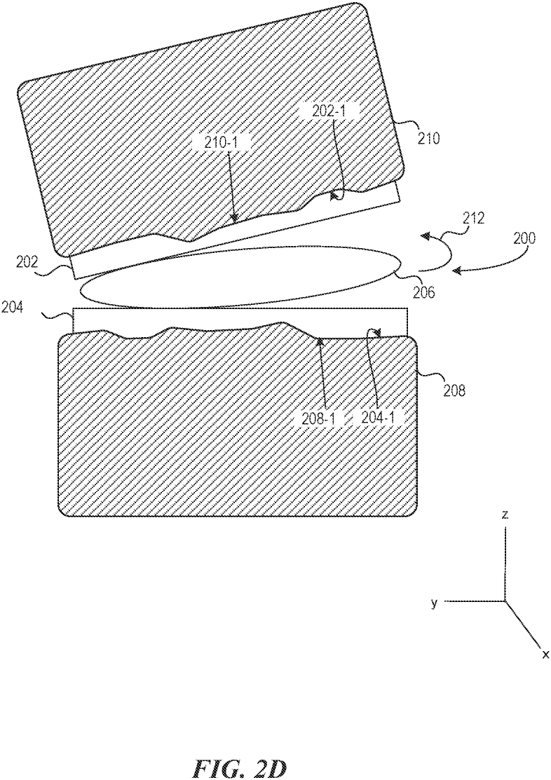

FIG. 2C is a side view of the device 200 implanted between the vertebral bodies 208, 210 and in a second configuration demonstrating a translation movement of the device 200, and FIG. 2D is a front view of the device 200 between the vertebral bodies 208, 210 in a third configuration demonstrating a rotational movement of the device 200. Referring first to FIG. 2C, the device 200 is in a configuration in which the first end-plate 202 (together with the vertebral body 210) has translated relative to the second end-plate 204 (and the vertebral body 208) in the x-direction, as indicated by an arrow 211. In FIG. 2D the device 200 is in a configuration in which the first end-plate 202 (together with the vertebral body 210) has rotated or pivoted relative to the second end-plate 204 (and the vertebral body 208) about the x-axis, as indicated by an arrow 212. Although only one translational movement and one rotational movement are illustrated in FIGS. 2C and 2D, the mobility element 206 may permit translational and/or rotational movements in other directions or planes, as previously described. Additional embodiments of mobility elements are illustrated in FIGS. 3-5.

FIG. 3 is a front view of another patient-specific arthroplasty system 300 (referred to as “the system 300”) positioned between vertebral bodies 326, 328, and 330 and configured in accordance with select embodiments of the present technology. In some embodiments, the system 300 corresponds to the system 100 described with respect to FIG. 1A. The system 300 includes two patient-specific arthroplasty devices: a first patient-specific arthroplasty device 302 (referred to as “the first device 302”) and a second patient-specific arthroplasty device 304 (referred to as “the second device 304”). In some embodiments, the first and second devices 302 and 304 correspond to the device 200 described with respect to FIGS. 2A-2D, except that devices 302 and 304 include mobility elements corresponding to hinge joints (e.g., hinge joints 305 and 307 in FIG. 3).

As shown, the first device 302 includes a first (e.g., upper or superior) end-plate 306 and a second (e.g., lower or inferior) end-plate 308. Likewise, the second device 304 includes a first (e.g., upper or superior) end-plate 310 and a second (e.g., lower or inferior) end-plate 312. The first end-plate 306 of the first device 302 is configured to engage an inferior surface of the vertebral body 326, and the second end-plate 308 of the first device 302 is configured to engage a superior surface of the vertebral body 328. The first end-plate 310 of the second device 304 is configured to engage an inferior surface of the vertebral body 328, and the second end-plate 312 is configured to engage a superior surface of the vertebral body 330. The first and second devices 302 and 304 are positioned to replace adjacent intervertebral discs. Alternatively, in some embodiments, the first and second devices 302 and 304 can be positioned so that there are one or more intervertebral discs between them that are not replaced with arthroplasty devices. Some or all of the end-plates 306, 308, 310, and 312 can include patient-specific topographies, as described with respect to FIGS. 2A-2D.

The first device 302 includes the hinge joint 305 corresponding to a mobility element. As described above, a mobility element of an arthroplasty device allows translational and/or rotational movement of the end-plates, and thus translational and/or rotational movement of the spine. In the illustrated embodiment, the hinge joint 305 includes a pin 320 coupled to/extending from the second end-plate 308. The pin 320 is positioned within a hinge 318 coupled to/extending from the first end-plate 306. In other embodiments, the pin 320 can extend from/be coupled to the first end-plate 306, and the hinge 318 can extend from/be coupled to the second end-plate 308. The hinge joint 305 allows the hinge 318 along with the first end-plate 306 (and the vertebral body 326) to pivot or rotate relative to the second end-plate 308 (and the vertebral body 328) about the x-axis (e.g., a left-right rotation), as shown by arrow 332. The hinge joint 305 may further allow translational movement of the first end-plate 306 (and the vertebral body 326) relative to the second end-plate 308 (and the vertebral body 328). For example, the hinge joint 305 may allow translational movement of the end-plate 306 relative to the end-plate 308 along the y-direction (e.g., side-to-side translation). In some embodiments, the hinge joint 305, therefore, corresponds to a semi-constrained mobile device, as described above.

The second device can include a hinge joint 307 that can be the same as or generally similar to the hinge joint 305 of the device 302. For example, the hinge joint 307 includes a pin 324 coupled to/extending from the second end-plate 312. The pin 324 is positioned within a hinge 322 coupled to/extending from the first end-plate 310. Similar to the hinge joint 305, the hinge joint 307 allows the hinge 322 (along with the first end-plate 310 and the vertebral body 328) to pivot or rotate relative to the second end-plate 312 and the vertebral body 330 about the x-axis (e.g., a left-right rotation), as shown by arrow 334. The hinge joint 307 may further allow translational movement of the first end-plate 310 (and the vertebral body 328) relative to the second end-plate 312 (and the vertebral body 330) (e.g., in the y-direction). The hinge joint 307 therefore also may correspond to a semi-constrained mobile device, as described above.

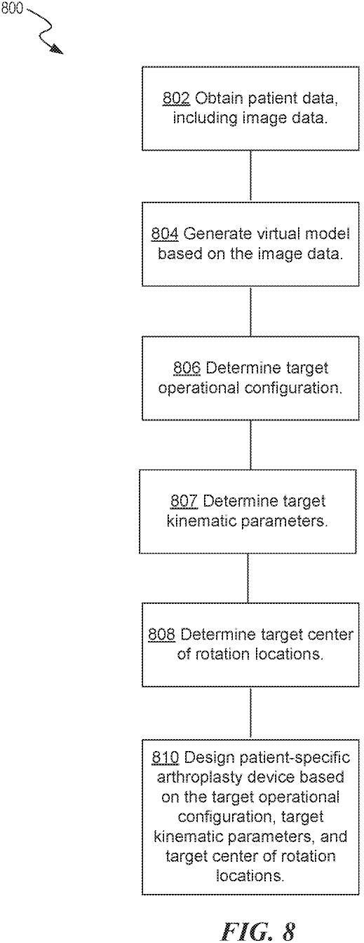

As shown, the hinge joints 305 and 307 are positioned at different positions with respect to a reference line R1 passing through the vertebral bodies 326, 328, and 330 in the z-direction. In some embodiments, the reference line R1 corresponds to a line passing through geometric centers of the vertebral bodies 326, 328, and 330 when the spinal segment formed by the vertebral bodies 326, 328, and 330 is in a rest position (e.g., the spinal segment is not in a rotational or translational state). As shown, the hinge joint 305 is positioned along the reference line R1 in FIG. 3 while the hinge joint 307 is positioned away from the reference line R1 by a distance (e.g., a distance D2). In some embodiments, the distance D2 is at least 0.1 cm, at least 0.2, at least 0.3, at least 0.4, at least 0.5, or at least 0.75 cm). As set forth in detail below, the position of a mobility element (e.g., relative to a geometric center of a respective arthroplasty device) can be determined using patient-specific metrics and designed to achieve an optimal post-surgical outcome. In some embodiments, the relative positions of the hinge joints 305 and 307 are pre-determined based on parameters associated with the patient, such as the patient’s anatomy, pathology, diagnosis, age, gender, activity level, health conditions, or the like. A method of designing arthroplasty devices and systems of the present disclosure are described in detail with respect to FIG. 8.

In some embodiments, the patient-specific arthroplasty devices of the present disclosure include one or more stoppers. For example, the first and second devices 302 and 304 can have first and second stoppers 314 and 316, respectively. The stoppers 314, 316 are positioned between respective end-plates of the first and second devices 302 and 304. In particular, the stoppers 314 are positioned between the first and second end-plates 306 and 308 of the first device 302, and the stoppers 316 are positioned between the first and second end-plates 310 and 312 of the second device 304.

The stoppers 314, 316 are configured to dampen and/or restrain movement (e.g., rotational and/or translation movement) of the end-plates. In particular, the stoppers 314, 316 are configured to dampen or restrain further movement of the end-plates toward each other when a distance between the end-plates 310 and 312 (e.g., a distance between peripheral areas of the end-plates) is below a threshold distance corresponding to a height of the stoppers 314, 316. The amount of dampening or restraining is pre-determined based on the operational target movement determined for a specific patient. For example, the one or more stoppers 314, 316 are positioned to ensure that the end-plates do not get in contact with each other and/or that the end-plates stay within a threshold distance from each other (e.g., the distance D1 illustrated in FIG. 2A is not less than a pre-determined threshold distance). The stoppers 314, 316 may be positioned at any position based on the target operative configuration for the region of the patient’s spine. For example, the stoppers 314, 316 can be positioned at pre-determined distances from the reference line R1 corresponding to a geometric center of the device. In some embodiments, the stoppers 314, 316 are positioned in a peripheral region of the arthroplasty device. Similarly, a size, shape, and/or other property (e.g., type of material or property of the material that a stopper is made of) of each stopper is determined based on the target operative configuration of the patient’s spine.

In some embodiments, a first device of an arthroplasty system (e.g., device 302 of system 300) includes one or more stoppers having a first size, shape and/or other property and a second device of an arthroplasty system (e.g., device 304 of system 300) includes one or more stoppers having a second size, shape and/or other property different from the first device. As shown in FIG. 3, the stoppers 316 of the second device 304 extend from of the first end-plate 310 to of the second end-plate 312 so that the stoppers 316 are in contact with both the first and second end-plates 310 and 312. In contrast, the stoppers 314 of the first device 302 extend only partially between the first and second end-plates 306 and 308 so that the stoppers 314 are in contact with the first end-plate 306 but not in contact with the second end-plate 308 when the vertebral bodies 326 and 328 are not pivoted or rotated (e.g., the spine is in a rest position). In some embodiments, an arthroplasty device can include two or more stoppers having different sizes, shapes and/or other properties.

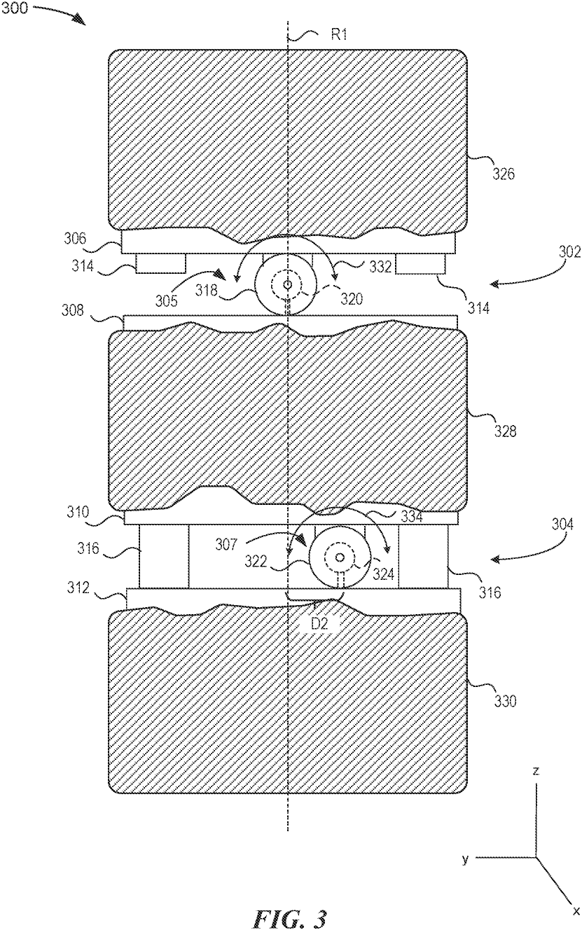

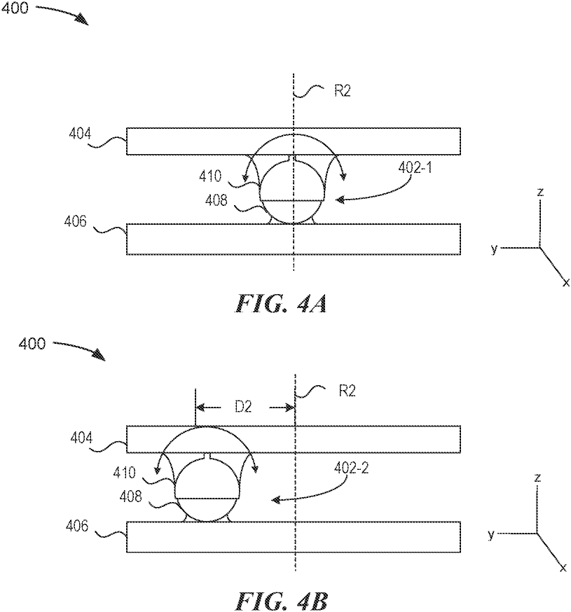

FIGS. 4A and 4B illustrate additional patient-specific arthroplasty devices configured in accordance with select embodiments of the present technology. In particular, FIG. 4A illustrates a first patient-specific arthroplasty device 400a (“the device 400a”) , and FIG. 4B illustrates a second patient-specific arthroplasty device 400b (“the device 400b”). In some embodiments, the devices 400a, b correspond to the device 200 described above with respect to FIGS. 2A-2D except that devices 400a, b include a mobility element corresponding to a ball-and-socket joint 402 (e.g., a ball-and-socket joint 402-1 in the first device 400a and a ball-and-socket joint 402-2 in the device 400b). The devices 400a, b each include first and second end-plates 404 and 406, e.g., corresponding to the first and second end-plates 202 and 204 described with respect to FIG. 2A. The ball-and-socket joint 402 is positioned between the first and second end-plates 404 and 406. As shown in FIG. 4A, the ball-and-socket joint 402 of the first device 400a is in a first position (e.g., indicated as ball-and-socket joint 402-1), and, as shown in FIG. 4B, the ball-and-socket joint 402 of the second device 400b is in a second position (e.g., indicated as ball-and-socket joint 402-2). As described above with respect to FIG. 3, a position of a mobility element (such as the ball-and-socket joint 402) with respect to a reference line (e.g., a reference line corresponding to a geometric center of the arthroplasty device) can be adapted based on patient-specific needs and requirements. in the embodiment illustrated in FIG. 4A, the ball-and-socket joint 402-1 is positioned along a reference line R2 (e.g., the reference line R2 corresponding to a geometric center of device 400) and in the embodiment illustrated in FIG. 4B, the ball-and-socket joint 402-2 is positioned away from reference line R2 by a distance D2.

Referring collectively to FIGS. 4A and 4B, the ball-and-socket joint 402 includes a ball 408 coupled to the second end-plate 406 and a round-shaped socket 410 coupled to the first end plate 404 and configured to mate with the ball 408. The ball-and-socket joint 402 allows for pivoting and/or rotating of the first end-plate 404 relative to the second end-plate 406 as the socket 410 rotates with respect to the ball 408 (e.g., in three dimensions). In some embodiments, the ball-and-socket joint 402 is further configured to translate between different positions along the surfaces of the end-plates (e.g., along the xy-plane in accordance with the xyz-coordinates). For example, in some embodiments the ball-and-socket joint 402 can be configured to transition between the position illustrated in FIG. 4A and the position illustrated in FIG. 4B, such that the FIGS. 4A and 4B illustrate two separate configurations of the same device, rather than different embodiments of arthroplasty devices. In such embodiments, the socket 410 and ball 408 are slidably coupled with the respective end-plates 404 and 406. In some embodiments, the ball-and-socket joint 402 further allows the first and second end-plates 404 and 406 to move translative relative to each other, as described above with respect to FIG. 2C. In such embodiments, the ball-and-socket joint 402 is an example of an unconstrained mobility element. As described above, an unconstrained mobility element allows a combination of rotational and translational movement of end-plates.

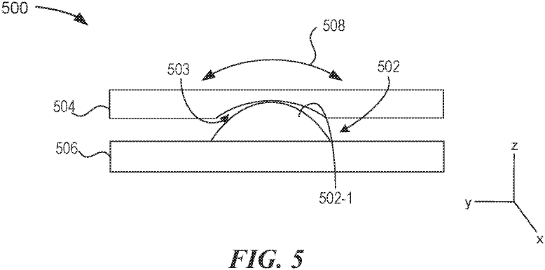

FIG. 5 is a schematic illustration of another patient-specific arthroplasty device 500 (referred to as “device 500”) configured in accordance with select embodiments of the present technology. The device 500 includes first and second end-plates 504 and 506, e.g., corresponding to the first and second end-plates 202 and 206 described with respect to FIG. 2A. The device 500 further includes a mobility element 502 having a dome-shaped element 502-1 extending from an inward-facing surface of the second end-plate 506. In some embodiments, the dome shaped element 502-1 is partially embedded inside a corresponding recess 503 within the first end-plate 504. The mobility element 502 allows for pivoting or rotating of the first end-plate 504 relative to the second end-plate 506 about the x-axis, as indicated by arrow 508.

As one skilled in the art will appreciate from the disclosure herein, the embodiments of FIG. 2A-5 are provided as simple schematic examples of patient-specific arthroplasty devices and systems of the present disclosure. Because the patient-specific arthroplasty devices described herein are designed to match individual patient anatomy, the size, shape, and geometry of the patient-specific arthroplasty devices will vary according to the individual patient’s anatomy. The present technology is thus not limited to any particular artificial device design or configuration, and can therefore include other devices beyond those illustrated or described herein.

As described in greater detail below with respect to FIG. 8, a patient-specific arthroplasty device (e.g., any of the devices 200, 302, 304, 400, and 500 described with respect to FIG. 2A-5) including two end-plates and a mobility element can be designed to have the appropriate orientation, rotation, flexion, and/or translation to enable a vertebral body (e.g., the vertebral body 210 shown in FIG. 2A) to move relative to another vertebral body (e.g., the vertebral body 208 shown in FIG. 2A) in accordance with one or more target kinematic parameters. The degree and type of motion permitted by the mobility element can be based on a number of factors, including, but not limited to, the composition of the mobility element, an interface between mated surfaces, and/or the geometry of the mobility element (e.g., type, contour, shape, diameter, etc.). The degree and type of motion permitted by the mobility element can further be based on age, gender, size, health conditions, activity level and/or other health-associated parameters of the patient. In some embodiments, the degree and type of motion permitted by the mobility element is facilitated by a design of material for the mobility element. For example, the mobility element can be made of any suitable materials including, but are not limited to, elastomeric polymers, rigid polymers, hybrid materials with elastomeric and rigid properties, ceramics, metals, and combinations thereof.

In some embodiments, the degree and type of motion permitted by the patient-specific arthroplasty device is facilitated by a design of the mobility element. For example, the mobility element described with respect to FIGS. 2A-2D corresponds to a double-curve-shaped core (e.g., the mobility element 206), the mobility element described with respect to FIG. 3 corresponds to an inner pin coupled with a hinge (e.g., the hinge joint 305 or 307), the mobility element described with respect to FIGS. 4A-4B corresponds to a ball-and-socket type joint, and the mobility element described with respect to FIG. 5 corresponds to a core having dome-shaped element that fits within a recess. Additionally, in some embodiments, the mobility element includes one or more biasing members, springs, sliding members/interfaces, or other elastic feature(s).

As one skilled in the art will appreciate, a patient-specific arthroplasty system (e.g., system 100 described with respect to FIG. 1A) may include two or more of the arthroplasty devices described with respect to FIG. 2A-5. In some embodiments, the two or more arthroplasty devices include arthroplasty devices of the same type (e.g., as illustrated in system 300 including the devices 302 and 304 with the hinge joints 305 and 307, respectively). In some embodiments, the two or more arthroplasty devices include a combination of different types of arthroplasty devices (e.g., a combination of one or more of the devices 200 and one or more of the devices 302, a combination of one or more of the devices 400 and one or more of the devices 500, a combination of one or more of the devices 200 and one or more of the devices 400, etc.).

As one skilled in the art will appreciate, in some embodiments the mobility element can be omitted and the end-plates can be configured to provide motion in the arthroplasty element. For example, the first end-plate 202 in FIGS. 2A-2D may form an interface (e.g., an articulating interface) with the second end-plate 204 that at least partially defines a motion segment in the implant. In such embodiments, the interface between the first end-plate 202 and the second end-plate 204 may be any suitable interface that permits movement between two components, including, but not limited to, a ball and socket interface, a dome and cup interface, a sliding interface, a rotating interface, etc. In some embodiments, the second (e.g., inward facing) surface 202-2 of the first end-plate 202 directly engages the second surface 204-2 of the second end-plate 204 to form the interface that defines the motion segment.

### Systems for Designing and Manufacturing Patient-Specific Arthroplasty Devices

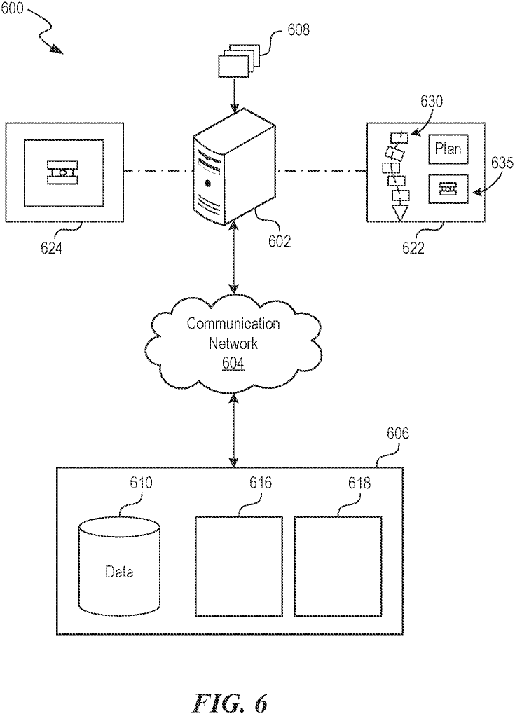

FIG. 6 is a network connection diagram illustrating a computing system 600 for providing patient-specific devices in accordance with embodiments of the present technology. The system 600 can include, among other things, a computing device 602, a communication network 604, a server 606, a display 622, and a manufacturing system 624. As described in greater detail below, the system 600 can be used to design patient-specific medical devices, such as patient-specific arthroplasty devices (e.g., implants) described herein, that fit native patient anatomy and/or a target operational configuration while also replicating and/or approximating the kinematics of a healthy or “normal” joint. Accordingly, in at least some embodiments, the system 600 can be used as part of a treatment plan for addressing damage by arthritis or other type of trauma resulting in the need for a joint replacement.

The computing device 602 can be a user device, such as a smart phone, mobile device, laptop, desktop, personal computer, tablet, phablet, or other such devices known in the art. As discussed further herein, the computing device 602 can include one or more processors, and memory storing instructions executable by the one or more processors to perform the methods described herein. The computing device 602 can be associated with a healthcare provider that is treating the patient. Although FIG. 6 illustrates a single computing device 602, in alternative embodiments, the computing device 602 can instead be implemented as a client computing system encompassing a plurality of computing devices, such that the operations described herein with respect to the computing device 602 can instead be performed by the computing system and/or the plurality of computing devices.

The computing device 602 is configured to obtain (e.g., receive, determine, etc.) a patient data set 608 associated with a patient to be treated. The patient data set 608 can include image data and/or kinematic data of the patient’s spine. Image data can include, for example, Magnetic Resonance Imaging (MRI) images, ultrasound images, Computerized Aided Tomography (CAT) scan images, Positron Emission Tomography (PET) images, X-Ray images (e.g., bi-planar radiography), camera images, and the like. The image data may show patient anatomy, such as the geometry, orientation, and topography of various anatomical features. In some embodiments, for example, the image data may show (and/or be used to determine) vertebral spacing, vertebral orientation, vertebral translation, abnormal bony growth, abnormal joint growth, joint inflammation, joint degeneration, tissue degeneration, stenosis, scar tissue, lumbar lordosis, Cobb angle(s), pelvic incidence, disc height, segment flexibility, rotational displacement, and other spinal tissue characteristics. Kinematic data can include, for example, specific values or other data corresponding to one or more kinematic parameters, such as values or other data corresponding to the range of motion in three dimensions (including, e.g., flexion, extension, bending, etc.), flexion/extension arcs, left/right bending arcs, lateral bending, angle of bend, angle of rotation, centers of rotation, displacement, and the like. The kinematic data can be obtained under a variety of conditions (e.g., load-bearing, non-load bearing, etc.). The values for the kinematic parameters can be determined based on images of the patient in different positions, measuring body position/motion, or the like. For example, characteristics of bony kinematic relationships can be determined by imaging the patient (e.g., X-ray, MRI, CAT scan, etc.) during movement, and analyzing the morphology of the patient based on the images. In some embodiments, the range of motion can be defined as a spherical range of motion, in which one vertebra moves relative to another vertebra in a spherical manner. In other embodiments, the range of motion can be defined as a relatively more complex range of motion defined by a three-dimensional curve through space. In some embodiments, and as described in greater detail below, the system 600 is configured to determine kinematic data based on the image data. In such embodiments, the patient data set 608 received by the computing device 602 does not necessarily include kinematic data.

In addition to image data and/or kinematic data, the patient data set 608 can include additional data including, but not limited to, medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health-related quality of life (HRQL)), vital signs, diagnostic results, medication information, allergies, diagnostic equipment information (e.g., manufacturer, model number, specifications, user-selected settings/configurations, etc.), or any combination of the foregoing. In some embodiments, the patient data set 608 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine. In some embodiments, the patient data set 608 further includes data representing the patient’s lifestyle such as level of activity, level of daily body movement, etc.

The computing device 602 can include or be operably coupled to a display 622 for providing output to a user (e.g., clinician, surgeon, healthcare provider, patient). In some embodiments, the display 622 can include a graphical user interface (GUI) for visually depicting a virtual model 630 of one or more regions of the patient’s anatomy based on the patient data set 608. The virtual model 630 can be a 2D model, a 3D model, CAD models, or other suitable models that provide a virtual representation of the patient’s anatomy. The one or more regions can include, but are not limited to, regions of the patient’s spine (e.g., cervical, thoracic, lumbar, and/or sacral). For example, in one embodiment, the target region may be a segment of the patient’s spine between C6 and C3. In such embodiments, the virtual model 630 may include individual vertebrae between C6 and C3 and other associated anatomical structures, such as discs between the vertebrae. In other embodiments, the virtual model may include a model of the patient’s entire spine (or generally the entire spine), rather than just specific segments. In some embodiments, generating the virtual model 630 from the image data includes reconstructing the two-dimensional image data containing pixels into three-dimensional volumetric data containing voxels that are representative of patient anatomy. In some embodiments, the image data and/or virtual model can be segmented to provide better viewing of individual anatomical features. The segmentable anatomical features can be any anatomy of interest, such as bones, discs, organs, joints, etc. In some embodiments, for example, the bony anatomy (e.g., vertebrae) are segmented from other anatomy to enable independent viewing of individual bony structures (e.g., vertebrae). In some embodiments, the display 622 can include a touch screen or other input module that permits a user to optionally manipulate the virtual model 630.

The computing device 602 can also be operably connected via a communication network 604 to a server 606, thus allowing for data transfer between the computing device 602 and the server 606. The communication network 604 may be a wired and/or a wireless network. The communication network 604, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 606, which may also be referred to as a “treatment assistance network” or “prescriptive analytics network,” can include one or more computing devices and/or systems. As discussed further herein, the server 606 can include one or more processors, and memory storing instructions executable by the one or more processors to perform the methods described herein. In some embodiments, the server 606 is implemented as a distributed “cloud” computing system or facility across any suitable combination of hardware and/or virtual computing resources.

The computing device 602 and server 606 can individually or collectively perform the various methods described herein for providing patient-specific medical care. For example, some or all of the steps of the methods described herein can be performed by the computing device 602 alone, the server 606 alone, or a combination of the computing device 602 and the server 606. Thus, although certain operations are described herein with respect to the server 606, it shall be appreciated that these operations can also be performed by the computing device 602, and vice-versa.

The server 606 includes at least one database 610 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 610 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient’s condition, anatomy, pathology, kinematics, medical history, preferences, and/or any other information or parameters relevant to the reference patient, such as any of the data described herein with respect to the patient data set 608. In some embodiments, the reference patient data set includes pre-operative data, intra-operative data, and/or postoperative data. For example, a reference patient data set can include data representing one or more of anatomy data, kinematic data, motion data, patient ID, age, gender, BMI, lumbar lordosis, Cobb angle(s), pelvic incidence, disc height, segment flexibility, bone quality, rotational displacement, and/or treatment level of the spine.

In some embodiments, the server 606 receives at least some of the reference patient data sets from a plurality of healthcare provider computing systems. Each healthcare provider computing system can include at least one reference patient data set (e.g., reference patient data sets) associated with reference patients treated by the corresponding healthcare provider. The reference patient data sets can include, for example, kinematic records, electronic medical records, electronic health records, biomedical data sets, etc.

As described in further detail herein, the server 606 can be configured with one or more algorithms that generate patient-specific treatment plan data (e.g., patient-specific treatment procedures, patient-specific implants) based on the reference data. In some embodiments, the patient-specific data is generated based on correlations between the patient data set 608 and the reference data. Optionally, the server 606 can predict outcomes, including recovery times, efficacy based on clinical end points, likelihood of success, predicted mortality, predicted related follow-up surgeries, or the like. In some embodiments, the server 606 can continuously or periodically analyze patient data (including patient data obtained during the patient stay) to determine near real-time or real-time risk scores, mortality prediction, etc.

In some embodiments, the server 606 includes one or more modules for performing one or more steps of the patient-specific treatment planning methods described herein. For example, in the depicted embodiment, the server 606 includes a data analysis module 616 and a treatment planning or implant design module 618. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The data analysis module 616 is configured with one or more algorithms for identifying a subset of reference data from the database 610 that is likely to be useful in developing a patient-specific treatment plan. For example, the data analysis module 616 can compare patient-specific data (e.g., the patient data set 608 received from the computing device 602) to the reference data from the database 610 (e.g., the reference patient data sets) to identify similar data (e.g., one or more similar patient data sets in the reference patient data sets). The comparison can be based on one or more parameters, such as age, gender, BMI, pathology, kinematics, lumbar lordosis, pelvic incidence, and/or treatment levels. The parameter(s) can be used to calculate a similarity score for each reference patient. The similarity score can represent a statistical correlation between the patient data set 608 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 616 can further be configured with one or more algorithms to select a subset of the reference patient data sets, e.g., based on similarity to the patient data set 608 and/or treatment outcome of the corresponding reference patient. For example, the data analysis module 616 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, range of motion, kinematic data, HRQL, activity level, complications, recovery times, efficacy, mortality, or follow-up surgeries. As described in further detail below, in some embodiments, the data analysis module 616 calculates an outcome score by assigning values to each outcome parameter. A patient can be considered to have a favorable outcome if the outcome score is above, below, or at a specified threshold value.

In some embodiments, the data analysis module 616 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 616 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular healthcare provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific treatment plan to perform a battlefield surgery can be based on reference patient data from similar battlefield surgeries and/or datasets associated with battlefield surgeries. In another example, the patient-specific treatment plan can be generated based on available robotic surgical systems. The reference patient data sets can be selected based on patients that have been operated on using comparable robotic surgical systems under similar conditions (e.g., size and capabilities of surgical teams, hospital resources, etc.).

The implant design module 618 is configured with one or more algorithms to generate at least one treatment plan (e.g., pre-operative plans, surgical plans, postoperative plans, etc.) and/or implant design based on, for example, the output from the data analysis module 616. In some embodiments, the implant design module 618 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 616 is analyzed (e.g., using statistics, machine learning, neural networks, AI, etc.) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a treatment plan will produce a favorable outcome for the particular patient. The predictive model(s) can be validated, e.g., by inputting data into the model(s) and comparing the output of the model to the expected output.

In some embodiments, the implant design module 618 is configured to generate the implant design based on previous treatment data from reference patients. For example, the implant design module 618 can receive a selected subset of reference patient data sets and/or similar patient data sets from the data analysis module 616, and determine or identify treatment data from the selected subset. The treatment data can include, for example, range of motion and/or other kinematic data, treatment procedure data (e.g., surgical procedure or intervention data) and/or medical device design data (e.g. implant design data) that are associated with favorable or desired treatment outcomes for the corresponding patient. The implant design module 618 can analyze the treatment procedure data and/or medical device design data to determine an optimal treatment protocol for the patient to be treated. For example, the treatment procedures and/or medical device designs can be assigned values and aggregated to produce a treatment score. The patient-specific treatment plan can be determined by selecting treatment plan(s) based on the score (e.g., higher or highest score; lower or lowest score; a score that is above, below, or at a specified threshold value). The personalized patient-specific treatment plan can be based on, at least in part, the patient-specific technologies or patient-specific selected technology.

Alternatively or in combination, the implant design module 618 can generate the implant designs based on correlations between data sets. For example, the implant design module 618 can correlate implant designs and medical device design data from implant designs for similar patients with favorable outcomes (e.g., as identified by the data analysis module 616). Correlation analysis can include transforming correlation coefficient values to values or scores. The values/scores can be aggregated, filtered, or otherwise analyzed to determine one or more statistical significances. These correlations can be used to determine treatment procedure(s) and/or medical device design(s) that are optimal or likely to produce a favorable outcome for the patient to be treated.

Alternatively or in combination, the implant design module 618 can generate designs using one or more AI techniques. AI techniques can be used to develop computing systems capable of simulating aspects of human intelligence, e.g., learning, reasoning, planning, problem-solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the implant design module 618 generates the treatment plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 610, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training dataset can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a treatment plan, the patient data set 608 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one treatment plan for the patient. In embodiments where multiple trained machine learning models are used, the models can be run sequentially or concurrently to compare outcomes and can be periodically updated using training data sets. The implant design module 618 can use one or more of the machine learning models based the model’s predicted accuracy score.

The patient-specific treatment plan generated by the implant design module 618 can include at least one patient-specific treatment procedure (e.g., a surgical procedure or intervention) and/or at least one patient-specific medical device (e.g., an implant or implant delivery instrument). A patient-specific treatment plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient. In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, discs, arthroplasty devices, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, or the like.

A patient-specific medical device design can include data representing one or more of physical properties (e.g., size, shape, volume, material, mass, weight,), mechanical properties (e.g., stiffness, strength, modulus, hardness, degrees of freedom of movement), and/or biological properties (e.g., osteo-integration, cellular adhesion, anti-bacterial properties, anti-viral properties) of a corresponding medical device. For example, a design for an orthopedic arthroplasty device can include implant shape, size, material, degrees of freedom of movement, center of rotation, etc. In some embodiments, the generated patient-specific medical device design is a design for an entire device (e.g., an arthroplasty device). Alternatively, the generated design can be for one or more components of a device (e.g., a mobility element of an arthroplasty device), rather than the entire device.

In some embodiments, the design is for one or more patient-specific device components that can be used with standard, off-the-shelf components. For example, in a spinal surgery, a pedicle screw kit can include both standard components and patient-specific customized components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient’s anatomy and/or surgeon’s preferences to enhance treatment. The patient-specific devices described herein are expected to improve delivery into the patient’s body, placement at the treatment site, and/or interaction with the patient’s anatomy.

In embodiments in which the patient-specific treatment plan includes a surgical procedure to implant a medical device, the implant design module 618 can also store various types of implant surgery information, such as implant parameters (e.g., types, dimensions), availability of implants, aspects of a pre-operative plan (e.g., initial implant configuration, detection, and measurement of the patient’s anatomy, etc.), FDA requirements for implants (e.g., specific implant parameters and/or characteristics for compliance with FDA regulations), or the like. In some embodiments, the implant design module 618 can convert the implant surgery information into formats useable for machine-learning based models and algorithms. For example, the implant surgery information can be tagged with particular identifiers for formulas or can be converted into numerical representations suitable for supplying to the trained machine learning model(s). The implant design module 618 can also store information regarding the patient’s anatomy, such as two- or three-dimensional images or models of the anatomy, and/or information regarding the biology, geometry, and/or mechanical properties of the anatomy. The anatomy information can be used to inform implant design and/or placement.

The treatment plan(s) generated by the implant design module 618 can be transmitted via the communication network 604 to the computing device 602 for output to a user (e.g., clinician, surgeon, healthcare provider, patient) via the display 622. As described previously, the display 622 can include a graphical user interface (GUI) for visually depicting various aspects of the treatment plan(s). For example, the display 622 can show various aspects of a surgical procedure to be performed on the patient, such as the surgical approach, treatment levels, corrective maneuvers, tissue resection, and/or implant placement. In addition to the virtual model 630 previously described, the display 622 can also show a design or rendering 635 of the patient-specific implant, such as a two- or three-dimensional model of the implant. The display 622 can also show patient information, such as two- or three-dimensional images or models of the patient’s anatomy where the surgical procedure is to be performed and/or where the device is to be implanted. The computing device 602 can further include one or more user input devices (not shown) allowing the user to modify, select, approve, and/or reject the displayed treatment plan(s).

In some embodiments, the medical device design(s) generated by the implant design module 618 can be transmitted from the computing device 602 and/or server 606 to a manufacturing system 624 for manufacturing a corresponding medical device. The manufacturing system 624 can be located on-site or off-site. On-site manufacturing can reduce the number of sessions with a patient and/or the time to be able to perform the surgery whereas off-site manufacturing can be useful for making the complex devices. Off-site manufacturing facilities can have specialized manufacturing equipment. In some embodiments, more complicated device components can be manufactured off-site, while simpler device components can be manufactured on-site.

Various types of manufacturing systems are suitable for use in accordance with the embodiments herein. For example, the manufacturing system 624 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 624 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 624 can manufacture one or more patient-specific medical devices based on fabrication instructions or data (e.g., CAD data, 3D data, digital blueprints, stereolithography data, or other data suitable for the various manufacturing technologies described herein). In some embodiments, the patient-specific medical device can include features, materials, and designs shared across designs to simplify manufacturing. For example, implants for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The treatment plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments, the implant design module 618 generates control instructions configured to cause a surgical robot (e.g., robotic surgery systems, navigation systems, etc.) to partially or fully perform a surgical procedure. The control instructions can be transmitted to the robotic apparatus by the computing device 602 and/or the server 606.

Following the treatment of the patient in accordance with the treatment plan, treatment progress can be monitored over one or more time periods to update the data analysis module 616 and/or implant design module 618. Post-treatment data can be added to the reference data stored in the database 610. The post-treatment data can be used to train machine learning models for developing patient-specific treatment plans, patient-specific medical devices, or combinations thereof.

It shall be appreciated that the components of the system 600 can be configured in many different ways. For example, in alternative embodiments, the database 610, the data analysis module 616 and/or the implant design module 618 can be components of the computing device 602, rather than the server 606. As another example, the database 610 the data analysis module 616, and/or the implant design module 618 can be located across a plurality of different servers, computing systems, or other types of cloud-computing resources, rather than at a single server 606 or computing device 602.

Additionally, in some embodiments, the system 600 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like. In some embodiments, the system 600 may include additional features and/or capabilities, such as any of those described in International Patent Application Publication No. WO2021/141849 or U.S. Pat. Application No. 16/987,113, the disclosures of which are incorporated by reference herein in their entireties.

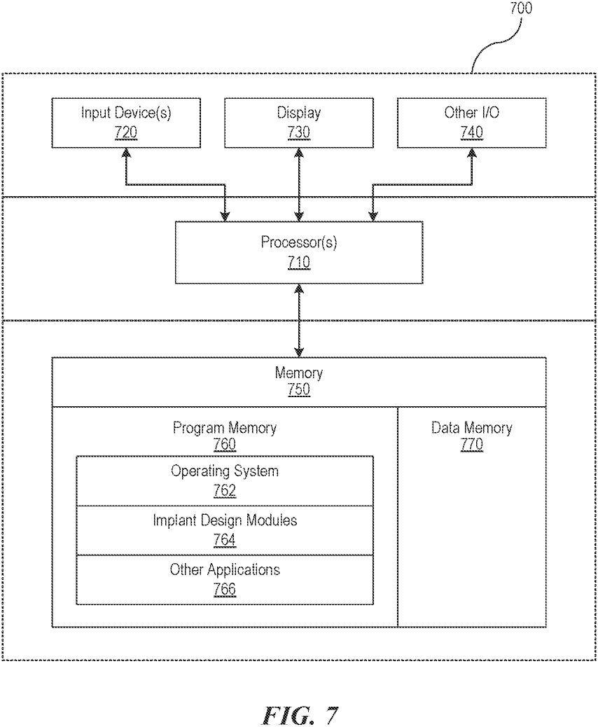

FIG. 7 illustrates a computing device 700 suitable for use in connection with the system 600 of FIG. 6 in accordance with select embodiments of the present technology. The computing device 700 can be incorporated in various components of the system 600 of FIG. 6, such as the computing device 602 or the server 606. The computing device 700 includes one or more processors 710 (e.g., CPU(s), GPU(s), HPU(s), etc.). The processor(s) 710 can be a single processing unit or multiple processing units in a device or distributed across multiple devices. The processor(s) 710 can be coupled to other hardware devices, for example, with the use of a bus, such as a PCI bus or SCSI bus. The processor(s) 710 can be configured to execute one more computer-readable program instructions, such as program instructions to carry out any of the methods described herein.

The computing device 700 can include one or more input devices 720 that provide input to the processor(s) 710, e.g., to notify it of actions from a user of one or more aspects of the computing device 700. The actions can be mediated by a hardware controller that interprets the signals received from the input device 720 and communicates the information to the processor(s) 710 using a communication protocol. Input device(s) 720 can include, for example, a mouse, a keyboard, a touchscreen, an infrared sensor, a touchpad, a wearable input device, a camera- or image-based input device, a microphone, or other user input devices.

The computing device 700 can include a display 730 used to display various types of output, such as text, models, virtual procedures, surgical plans, implants, graphics, and/or images (e.g., images with voxels indicating radiodensity units or Hounsfield units representing the density of the tissue at a location). For example, in some embodiments, the display 730 provides a two or three-dimensional virtual model of a patient’s spine. In some embodiments, the display 730 provides graphical and textual visual feedback to a user. The processor(s) 710 can communicate with the display 730 via a hardware controller for devices. In some embodiments, the display 730 includes the input device(s) 720 as part of the display 730, such as when the input device(s) 720 include a touchscreen or is equipped with an eye direction monitoring system. In alternative embodiments, the display 730 is separate from the input device(s) 720. Examples of display devices include an LCD display screen, an LED display screen, a projected, holographic, or augmented reality display (e.g., a heads-up display device or a head-mounted device), and so on. In some embodiments, the display 730 is configured to display a virtual model of a patient’s spine generated based on received patient data (e.g., image data), as previously described with respect to display 222.

Optionally, other I/O devices 740 can also be coupled to the processor(s) 710, such as a network card, video card, audio card, USB, firewire or other external devices, camera, printer, speakers, CD-ROM drive, DVD drive, disc drive, or Blu-Ray device. Other I/O devices 740 can also include input ports for information from directly connected medical equipment such as imaging apparatuses, including MRI machines, X-Ray machines, CT machines, etc. Other I/O devices 740 can further include input ports for receiving data from these types of machines from other sources, such as across a network or from previously captured data, for example, stored in a database.

In some embodiments, the computing device 700 also includes a communication device (not shown) capable of communicating wirelessly or wire-based with a network node. The communication device can communicate with another device or a server through a network using, for example, TCP/IP protocols. The computing device 700 can utilize the communication device to distribute operations across multiple network devices, including imaging equipment, manufacturing equipment, etc.

The computing device 700 can include memory 750, which can be in a single device or distributed across multiple devices. Memory 750 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy discs, CDs, DVDs, magnetic storage devices, tape drives, device buffers, and so forth. A memory is not a propagating signal divorced from underlying hardware; a memory is thus non-transitory. In some embodiments, the memory 750 is a non-transitory computer-readable storage medium that stores, for example, programs, software, data, or the like. In some embodiments, memory 750 can include a program memory 760 that stores programs and software, such as an operating system 762, one or more implant design modules 764, and other application programs 766. The implant design module(s) 764 can include one or more modules configured to perform the various methods described herein. Memory 750 can also include data memory 770 that can include, e.g., reference data, configuration data, settings, user options or preferences, etc., which can be provided to the program memory 760 or any other element of the computing device 700.

### Methods for Designing Patient-Specific Devices

FIG. 8 is a flow diagram illustrating a method 800 for designing a patient-specific arthroplasty device or a system including two or more patient-specific arthroplasty devices in accordance with select embodiments of the present technology (e.g., designing any of the systems and devices described with respect to FIG. 2A-5).

The method 800 can begin in step 802 by obtaining patient data. Patient data can include image data and kinematic data of the patient’s spine. Image data can include, for example, Magnetic Resonance Imaging (MRI) images, ultrasound images, Computerized Aided Tomography (CAT) scan images, Positron Emission Tomography (PET) images, X-Ray images (e.g., bi-planar radiography), camera images, and the like. The image data may show the patient’s native anatomical configuration (e.g., pre-operative anatomy), such as the geometry, orientation, and topography of various anatomical features. In some embodiments, for example, the image data may show (and/or be used to determine) vertebral spacing, vertebral orientation, vertebral translation, centers of rotation, abnormal bony growth, abnormal joint growth, joint inflammation, joint degeneration, tissue degeneration, stenosis, scar tissue, lumbar lordosis, Cobb angle(s), pelvic incidence, disc height, segment flexibility, rotational displacement, and other spinal tissue characteristics.

Kinematic data can include, for example, specific values or other data corresponding to one or more kinematic parameters, such as values or other data corresponding to the range of motion in three dimensions (including, e.g., flexion, extension, bending, etc.), a center of rotation (COR) corresponding to the range of motion in the three dimensions, flexion/extension arcs, left/right bending arcs, lateral bending, angle of bend, angle of rotation, displacement, axial rotation, and the like. The kinematic data can be obtained under a variety of conditions (e.g., load-bearing, non-load bearing, etc.). In some embodiments, the range of motion can be defined as a spherical range of motion, in which one vertebra moves relative to another vertebra in a spherical manner. In other embodiments, the range of motion can be defined as a relatively more complex range of motion defined by a three-dimensional curve through space. Other patient data in addition to image data and kinematic data can optionally be obtained in step 802. Additional patient data can include, but is not limited to, medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health-related quality of life (HRQL)), vital signs, diagnostic results, medication information, allergies, and/or any combination of the foregoing.

In some embodiments, obtaining the kinematic data in step 802 includes determining the values of the one or more kinematic parameters using one or more software modules (e.g., the implant design module 618 in FIG. 6 and/or the implant design module 764 in FIG. 7). The software module may perform a kinematic evaluation of the patient based on the image data and/or other patient data to estimate various kinematic parameters for the patient. For example, the software module can analyze one or more anatomical features/measurements in the image data and/or virtual model to define kinematic parameters, determine kinematic relationships, and/or estimate various kinematic parameters. Suitable anatomical features/measurements include, but are not limited to, the distance between anatomical landmarks, fiducials, vertebral spacing, vertebral orientation, abnormal bony growth, abnormal joint growth, joint inflammation, joint degeneration, tissue degeneration, stenosis, scar tissue, and combinations thereof. Other patient data that can be used by the software module to estimate the kinematic parameters includes, but is not limited to, medical history, sex, age, height, weight, and the like. As a non-limiting example, the one or more software modules can automatically determine or at least estimate one or more centers of rotation of the patient’s spine based on the image data. In some embodiment, determining the optimal COR location is done by estimating an average COR for the different rotational motions. As described above, the CORs for specific vertebral bodies are patient-specific and depend on, e.g., the patient’s anatomy, age, size, activity, the patient’s health conditions, and/or other parameters associated with the patient’s spine.

In some embodiments, the software module may incorporate one or more artificial intelligence architectures for determining the various kinematic parameters based on the image data. The artificial intelligence architectures can be similar to those previously described herein, and can include, for example, trained neural networks (e.g., trained convolutional neural networks, etc.) for analyzing two-dimensional images and/or three-dimensional models. Without being bound by theory, using the one or more software modules to perform the kinematic evaluation can reduce and/or eliminate the need to conduct a manual evaluation of the patient’s kinematics before the implant surgery. However, in at least some embodiments, the kinematic data can be obtained through one or more standard kinematic studies. Therefore, in at least some embodiments, obtaining the kinematic data in step 802 includes receiving the values of the one or more kinematic parameters. For example, the values of the one or more kinematic parameters can be obtained from a motion study and inputted into a system performing the method 800 (e.g., inputted into the computing device 700 via the input device(s) 720, shown in FIG. 7) or through joint morphology studies.

The method 800 further includes generating, based at least in part on the image data obtained in step 802, a virtual model of one or more regions of the patient’s anatomy in step 804. The virtual model can be a 2D model, a 3D model, CAD models, or other suitable models that provide a virtual representation of the patient’s native anatomy. The one or more regions can include, but are not limited to, regions of the patient’s spine (e.g., cervical, thoracic, lumbar, and/or sacral). For example, in one embodiment, the target region may be a segment of the patient’s spine between C3 and C5. In such embodiments, the virtual representation may include individual vertebrae between C3 and C5 and other associated anatomical structures, such as discs between the vertebrae. In some embodiments, the virtual model may include a model of the patient’s entire spine, rather than just specific segments. In some embodiments, generating the virtual model from the image data includes reconstructing the two-dimensional image data containing pixels into three-dimensional volumetric data containing voxels that are representative of patient anatomy. In some embodiments, the image data and/or virtual model can be segmented to provide better viewing of individual anatomical features. The segmentable anatomical features can be any anatomy of interest, such as bones, discs, organs, etc. In some embodiments, for example, the bony anatomy (e.g., vertebrae) are segmented from other anatomy to enable independent viewing of individual bony structures (e.g., vertebrae). The virtual model can optionally be displayed to a physician, such as via the display 622, shown in FIG. 6. In some embodiments, step 804 is omitted and the method 800 proceeds directly from step 802 to step 806. In some embodiments, the virtual model may illustrate the determined or estimated centers of rotation for the displayed vertebral segment (e.g., as shown in FIG. 1B).

In step 806, a user (e.g., a surgeon or other physician) and/or a software module (e.g., the implant design module 618 and/or the implant design module 764) determines a target operational configuration for the one or more regions of the patient’s anatomy, which corresponds to a target post-surgical anatomical configuration. The target operational configuration can be different than the native anatomical configuration shown in the image data. The target operational configuration can include an adjustment to one or more anatomical features relative to the native anatomical configuration, including, but not limited to, an adjustment to the spacing between vertebral bodies, the orientation of vertebral bodies, alignment of two or more vertebral bodies, lumbar lordosis, Cobb angle(s), pelvic incidence, disc height, segment flexibility, rotational displacement, and the like. For example, in embodiments in which the patient has vertebral disc degeneration between two vertebrae, the image data may illustrate that the native anatomical configuration has a reduced or sub-optimal distance between an inferior boundary of a first vertebra and a superior boundary of the second vertebra. The target operational configuration may therefore include an increased distance between the first and second vertebrae that is reflective of a “healthy” or “normal” anatomy. In another example, the image data may illustrate that a first vertebra is out of alignment with a second vertebra. In such embodiments, the target operational configuration may therefore include realigning the first vertebra and the second vertebra.

In embodiments in which a user determines the target operational configuration, the user can use the virtual model to manipulate one or more relationships (distances, angles, constraints, etc.) between individual vertebrae to set the target operational configuration. Manipulations can include, but are not limited to, translation along an axis or curve, rotation about an axis or centroid, and/or rotation about the center of mass. In some embodiments, the manipulation can be done until the virtual model illustrates the anatomy in a “desired” anatomical configuration. The user can then provide an input setting the illustrated desired anatomical configuration as the target operational configuration.

In embodiments in which a software module determines the target operational configuration, the software module may automatically manipulate the virtual model to provide a recommended target operational configuration based on one or more design criteria and/or reference patient data sets. Suitable design criteria can include, for example, target values associated with various anatomical features, including, for example, target values associated with vertebral spacing (e.g., minimum vertebral body spacing, maximum vertebral spacing, etc.), vertebral orientation, vertebral alignment, vertebral translation, lumbar lordosis, Cobb angle(s), pelvic incidence, disc height, segment flexibility, rotational displacement, kinematics, or the like. Suitable reference patient data sets can be identified using, for example, the data analysis module 616 described previously with reference to FIG. 6. The implant design module can further perform one or more simulations, analyses (e.g., stress analysis, fatigue analysis, etc.), or the like to provide feedback (e.g., identified high-stress regions), design recommendations, treatment recommendations (e.g., steps to prepare implantation site), or the like. The software module used in step 806 to manipulate the virtual model and provide a recommended target operational configuration can be the same as or different than the software module used in step 802 to conduct the kinematic evaluation. In some embodiments, determining the target operational configuration includes using the software module to provide a recommended target operational configuration, and then permitting the physician to optionally further modify the target operational configuration.

The method 800 continues in step 807 by determining target (e.g., post-surgical) kinematic parameters. This can include, for example, determining the type (e.g., translational, rotational, etc.) and degree (e.g., magnitude) of movement for each vertebral segment that should be permitted. In some embodiments, this includes determining whether the patient’s symptoms will be best alleviated by permitting zero, one, two, three, four, five, or six degrees of freedom of movement.

In some embodiments, determining whether the post-surgical kinematic parameters should be different than the pre-surgical kinematic parameters includes identifying a reference data set of patients having similar pre-surgical conditions as the particular patient. The various post-surgical outcomes for the patients included in the reference data set can then be analyzed (e.g., using the software modules and/or artificial intelligence architectures described herein) to select target or optimal post-surgical kinematic parameters that are associated with the highest probability of a successful surgical outcome. Additional details for determining target kinematic parameters are described in U.S. Application No. 16/987,113, previously incorporated by reference herein.

The method 800 continues by determining target (e.g., post-surgical) COR locations in step 808 for the patient’s spine based on, for example, the generated virtual model, the target operational configuration determined in step 806, and/or the target kinematic parameters determined in step 807. In some embodiments, determining the target COR locations includes determining an optimal COR for each vertebral body within a region of the spine (e.g., the cervical segment of the spine). In some embodiments, determining the target COR locations includes determining the target CORs for different rotational motions (e.g., flexion-extension motion and left-right rotational motion as described with respect to FIG. 1B).

In some embodiments, the target CORs can be different than the CORs associated with the patient’s native (e.g., pre-surgical) anatomical configuration. For example, review of the virtual model may determine that the patient will experience less post-surgical pain and/or improved range of motion if the post-surgical COR is adjusted relative to the patient’s pre-surgical COR. In such embodiments, the methods may include selecting a target COR that is offset (e.g., at least by 0.1 cm, 0.2 cm, 0.3 cm, 0.4 cm, etc.) from the pre-surgical COR. Without being bound by theory, selecting target CORs based on optimized patient outcomes, as opposed to CORs based on the patient’s native, pre-surgical anatomical configuration, is expected to improve patient outcomes in arthroplasty procedures.

In some embodiments, determining whether the post-surgical COR should be different than the pre-surgical COR includes identifying a reference data set of patients having similar pre-surgical conditions as the particular patient. The various post-surgical outcomes for the patients included in the reference data set can then be analyzed (e.g., using the software modules and/or artificial intelligence architectures described herein) to select target or optimal post-surgical CORs that are associated with the highest probability of a successful surgical outcome.

Once the target operational configuration, target kinematic parameters, and the target COR locations are determined in steps 806, 807, and 808, respectively, the method 800 continues by designing a patient-specific arthroplasty device in step 810 configured to achieve the target operational configuration, the target kinematic parameters, and the target CORs. The patient-specific arthroplasty device can be designed using the software module, which can be the same as or different than the software modules optionally used in steps 802, 806, 807, and/or 808. To achieve the target operational configuration, the software module can design the patient-specific arthroplasty device to fit in the negative space (e.g., the “implant envelope”) of the target operational configuration, e.g., as displayed on the virtual model representing the target operational configuration. The negative space can be used to determine various geometric parameters of the patient-specific arthroplasty device. The geometric parameters include, but are not limited to, dimensions, heights, surfaces, topographies, footprints, and the like. In some embodiments, a virtual patient-specific arthroplasty device can be created and shown within the negative space of the virtual representation of the patient anatomy before the actual physical device is fabricated.

The software module also designs the patient-specific arthroplasty device to (1) achieve the target kinematic parameters determine in step 807, and (2) provide the target post-surgical CORs of the target region of the patient’s spine determined in step 808. For example, the software module can design various features of the arthroplasty devices, such as design features associated with the end-plates and/or the mobility element, to achieve the target CORs and/or the desired amount or type of motion. The design features associated with the end-plates include the type of materials used for the end-plates, attachment mechanisms for securing the end-plates to the respective vertebral bodies, and other features described with reference to FIG. 2A-5. The design features associated with the mobility element of the arthroplasty device includes the type of materials used for the mobility element, the position of the mobility element (e.g., relative to a geometric center of the arthroplasty device), and/or the type of the mobility element (e.g., the mobility elements described with respect to FIG. 2A-5), and other features described with reference to FIG. 2A-5. For example, the mobility element can be made of a ceramic material, polymeric material, metallic material, or a combination thereof. The position of the mobility element relative to the end plates can also be pre-determined based on the target COR.

In some embodiments, the COR is fixed to a position relative to the geometric center of the arthroplasty device. For example, the ball-and-socket joint 402 of arthroplasty device 400 described with respect to FIGS. 4A and 4B have fixed positions with respect to the reference line R2 (e.g., R2 corresponds to the geometric center of the device 400). In such embodiments, the mobility element is constrained in that the mobility element allows for rotational movement of the end-plates with respect to each other but does not allow for translational movement of the end-plates with relative each other. For example, in FIGS. 4A and 4B the ball-and-socket joint 402 may be coupled (fixed) to end-plates 404 and 406. In some embodiments, the COR is mobile reflecting typical anatomical movements of an intact disc. In such embodiments, the arthroplasty device is unconstrained in that the mobility element allows for rotational as well as the translational movement of the end-plates relative to each other. For example, in FIG. 2D, the mobility element 206 allows the first end-plate 202 to pivot or rotate with respect to the second end-plate 204 and in FIG. 2D the mobility element 206 allows the first end-plate 202 to translate (e.g., in the x direction and the y direction) relative to the second end-plate 204. The COR is mobile with respect to the geometric center of the arthroplasty device.

In some embodiments, the software module designs the patient-specific arthroplasty device to permit compression and decompression of the patient’s spine. The target post-surgical compression and decompression can also be pre-determined based on the analyses of steps 802 through 808 (e.g., determined as part of the analysis performed in step 807, since the compression and decompression represent magnitude of movement in the z-direction). The features of the arthroplasty devices, such as features related to stoppers (e.g., stoppers 314 and 316 in FIG. 3) and/or mobility elements (e.g., any mobility elements described with respect to FIG. 2A-5) are designed to allow the distance between the end-plates of the arthroplasty device to adapt (e.g., the distance D1 in FIG. 2A). The features related to the stoppers include, but are not limited to, a number of stoppers, relative positions of the stoppers, sizes, and shapes of the stoppers, and materials that the stoppers are made of (e.g., the elasticity of the materials). The features related to the mobility elements include, but are not limited to, a type, size, and relative positions of the mobility elements, and materials that the mobility elements are made of.

Various types of manufacturing systems are suitable for use in accordance with the embodiments herein. For example, the manufacturing system can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system can manufacture one or more patient-specific medical devices based on fabrication instructions or data (e.g., CAD data, 3D data, digital blueprints, stereolithography data, or other data suitable for the various manufacturing technologies described herein). In some embodiments, the patient-specific devices can include features, materials, and designs shared across designs to simplify manufacturing. For example, deployable patient-specific devices for different patients can have similar internal deployment mechanisms but have different deployed configurations. In some embodiments, the components of the patient-specific devices are selected from a set of available pre-fabricated components and the selected pre-fabricated components can be modified based on the fabrication instructions or data.

In combination with any of the above methods, the systems and methods described herein can also generate a medical treatment plan for a patient in addition to designing a patient-specific device. The medical treatment plan can include surgical information, surgical plans, technology recommendations (e.g., device and/or instrument recommendations), in addition to the medical device designs. For example, the medical treatment plan can include at least one treatment procedure (e.g., a surgical procedure or intervention) for implanting the patient-specific device. The systems described herein can be configured to generate a medical treatment plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disc, a hard disc drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. Application No. 16/048,167, filed on Jul. 27, 2018, titled “SYSTEMS AND METHODS FOR ASSISTING AND AUGMENTING SURGICAL PROCEDURES”;
U.S. Application No. 16/242,877, filed on Jan. 8, 2019, titled “SYSTEMS AND METHODS OF ASSISTING A SURGEON WITH SCREW PLACEMENT DURING SPINAL SURGERY”;
U.S. Application No. 16/207,116, filed on Dec. 1, 2018, titled “SYSTEMS AND METHODS FOR MULTI-PLANAR ORTHOPEDIC ALIGNMENT”;
U.S. Application No. 16/352,699, filed on Mar. 13, 2019, titled “SYSTEMS AND METHODS FOR ORTHOPEDIC DEVICE FIXATION”;
U.S. Application No. 16/383,215, filed on Apr. 12, 2019, titled “SYSTEMS AND METHODS FOR ORTHOPEDIC DEVICE FIXATION”;
U.S. Application No. 16/569,494, filed on Sep. 12, 2019, titled “SYSTEMS AND METHODS FOR ORTHOPEDIC DEVICES”;
U.S. Application No. 16/699,447, filed on Nov. 29, 2019, titled “SYSTEMS AND METHODS FOR ORTHOPEDIC DEVICES”;
U.S. Application No. 17/085,564, filed on Oct. 30, 2020, titled “SYSTEMS AND METHODS FOR DESIGNING ORTHOPEDIC DEVICES BASED ON TISSUE CHARACTERISTICS”;
U.S. Application No. 16/735,222, filed Jan. 6, 2020, titled “PATIENT-SPECIFIC MEDICAL PROCEDURES AND DEVICES, AND ASSOCIATED SYSTEMS AND METHODS”;
U.S. Application No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS”;
U.S. Application No. 17/100,396, filed Nov. 20, 2020, titled “PATIENT-SPECIFIC VERTEBRAL DEVICES WITH POSITIONING FEATURES”;
U.S. Application No. 17/342,439, filed Jun. 8, 2021, titled “PATIENT-SPECIFIC MEDICAL PROCEDURES AND DEVICES, AND ASSOCIATED SYSTEMS AND METHODS”; and
International Application No. PCT/US2021/044878, filed Aug. 6, 2021, titled “PATIENT-SPECIFIC ARTIFICIAL DISCS” IMPLANTS AND ASSOCIATED SYSTEMS AND METHODS.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

I/We claim:

1. A computer-implemented method for designing a patient-specific arthroplasty device, the method comprising:
obtaining patient data associated with one or more regions of a patient’s spine;
generating a virtual model of the one or more regions of the patient’s spine based on the patient data;
determining, based on the patient data and/or the virtual model, (1) a target post-surgical anatomical configuration for the one or more regions of a patient’s spine, (2) one or more target post-surgical kinematic parameters for the one or more regions of the patient’s spine, the one or more target post-surgical kinematic parameters including a target post-surgical type of motion and/or degree of motion, and (3) one or more target post-surgical centers of rotation for the one or more regions of the patient’s spine; and
designing the patient-specific arthroplasty device based on the target post-surgical anatomical configuration, the target post-surgical kinematic parameters, and the target post-surgical centers of rotation.

2. The method of claim 1, wherein the patient-specific arthroplasty device comprises:
a first end-plate having a first patient-specific topography;
a second end-plate having a second patient-specific topography; and
a mobility element disposed between the first end-plate and the second end-plate, wherein the mobility element is designed to comply with the one or more target post-surgical kinematic parameters and to achieve the target post-surgical center of rotation when the patient-specific arthroplasty device is implanted in the patient’s spine.

3. The method of claim 2, wherein the mobility element is designed for allowing
(1) rotation of the first end-plate and the second end-plate relative to each other, and (2) translation of the first end-plate and the second end-plate relative to each other.

4. The method of claim 3, wherein the mobility element is designed for allowing six degrees of freedom of movement between the first end-plate and the second end-plate.

5. The method of claim 1, wherein the patient data includes image data depicting a native anatomical configuration of the one or more regions of the patient’s spine.

6. The method of claim 1, wherein the patient data includes kinematic data associated with the one or more regions of a patient’s spine, wherein the kinematic data includes values for one or more kinematic parameters.

7. The method of claim 1, wherein at least one of the target post-surgical centers of rotation is offset from a geometric centerpoint between adjacent vertebral bodies in the one or more regions of the patient’s spine.

8. The method of claim 1, wherein the one or more target post-surgical centers of rotation are different than corresponding pre-surgical centers of rotation.

9. The method of claim 1, wherein the one or more target post-surgical kinematic parameters are different than corresponding pre-surgical kinematic parameters.

10. The method of claim 1 wherein the one or more target post-surgical kinematic parameters includes a target degree of rotation.

11. An arthroplasty system, comprising:
a patient-specific arthroplasty device for implantation in a patient’s spine, the patient-specific arthroplasty device comprising:
a first end-plate having a first patient-specific topography;
a second end-plate having a second patient-specific topography; and
a mobility element disposed between the first end-plate and the second end-plate, wherein:
the mobility element is configured to enable translational and rotational movement of the first end-plate and the second end-plate relative to each other, and
a position of the mobility element relative to the first end-plate and the second end-plate is designed to achieve a target center of rotation when the patient-specific arthroplasty device is implanted in the patient’s spine, wherein the target center of rotation is based at least in part on a target configuration for a region of the patient’s spine at which the patient-specific arthroplasty device is to be implanted.

12. The system of claim 11, wherein the mobility element includes a ball coupled to the first end-plate and a socket coupled to the second end-plate, the ball configured to mate with the socket, thereby allowing rotation of the first end-plate with respect to the second end-plate.

13. The system of claim 11, wherein the mobility element is offset from a geometric center of at least one of the first end-plate and the second end-plate.

14. The system of claim 13, wherein the mobility element is offset from a geometric center of both the first end-plate and the second end-plate.

15. The system of claim 13, wherein the mobility element is offset from the geometric center of the first end-plate and/or the second end-plate by at least 0.1 cm.

16. The system of claim 11, wherein the mobility element includes a hinge coupled to the first end-plate and a pin coupled to the second end-plate, wherein the pin is disposed within the hinge allowing rotation of the first end-plate with respect to the second end-plate.

17. The system of claim 11, wherein the mobility element is configured for allowing (1) at least two rotational degrees of freedom between the first end-plate and the second end-plate, and (2) at least two translational degrees of freedom between the first end-plate and the second end-plate.

18. The system of claim 11, wherein the mobility element is at least partially compressible such that a distance between the first end-plate and the second end-plate can be adaptable.

19. The system of claim 11, wherein the mobility element includes a ceramic, polymeric, metallic, or viscoelastic material.

20. The system of claim 11, further including:
one or more stoppers coupled with the first end-plate and disposed between the first end-plate and the second end-plate, wherein:
the one or more stoppers are positioned at a peripheral area of the first end-plate, and
the one or more stoppers are configured to dampen the movement of the first end-plate relative to the second end-plate when a distance between the peripheral area of the first end-plate and the second end-plate is below a threshold distance.

21. The system of claim 11, wherein the first end-plate has a first surface having the first patient-specific topography, the first-surface being configured to mate with a first vertebra to form a first generally gapless interface therebetween, and wherein the second end-plate has a second surface having the second patient-specific topography, the second surface being configured to mate with a second vertebra to form a second generally gapless interface therebetween.

22. The system of claim 11, wherein the first end-plate and the second end-plate are coupled to respective vertebrae by one or more of a keel, a spike, and a screw.

23. The system of claim 11, wherein the patient-specific arthroplasty device is a first patient specific arthroplasty device, the system further comprising a second patient-specific arthroplasty device, wherein the second patient-specific arthroplasty device is configured to be implanted within a separate intervertebral space relative to the first patient-specific arthroplasty device.

24. The system of claim 23, wherein the first patient-specific arthroplasty device is configured to achieve a first center of rotation at the corresponding intervertebral space, and wherein the second patient-specific arthroplasty device is configured to achieve a second center of rotation at the corresponding intervertebral space, the first and second centers of rotation being longitudinally offset.
